(19) United States
(12) Patent Application Publication
(10) Pub. No.: US 2026/0241151 A1
(43) Pub. Date: Aug. 20, 2026

(54) LIQUID APPLICATOR DEVICE

(71) Applicant: HEALTHIUM MEDTECH LIMITED, Bangalore (IN)

(72) Inventors: Michael RODRIGUES, Tamil Nadu Coimbatore (IN); Rajkumar THANGARAJ, Tamil Nadu Coimbatore (IN); Anand VENKATACHALAM, Tamil Nadu Coimbatore (IN); Jagadeeswaran K, Karnataka Bangalore (IN)

(21) Appl. No.: 19/162,064

(22) PCT Filed: Mar. 7, 2024
(86) PCT No.: PCT/IB2024/052199

§ 371 Date: Sep. 4, 2025

(30) Foreign Application Priority Data
Mar. 9, 2023 (IN) 202341015660

(51) Int. Cl. A61M 35/00

(52) U.S. Cl. CPC A61M 35/006; A61M 2205/3334; A61M 2207/00

(57) ABSTRACT

The present invention relates to a liquid applicator device (100) to apply/paint antiseptic liquid on a body part of a user The liquid applicator device (100) includes a storage unit (102) adapted to store liquid therein. The liquid applicator device (100) further includes a connecting unit (106) coupled with the storage unit (102). The liquid applicator device (100) further includes a regulating unit (104) coupled to the connecting unit (106) though an opening (210) of the connecting unit (106). The regulating unit (104) includes a spigot (212), first through hole (214), and a second through hole (216), adapted to regulate flow of the liquid in a controlled manner. The liquid applicator device (100) further includes an applicator (108) coupled to the connecting unit (106), adapted to absorb/retain the liquid for a predefined period and desorb the liquid when the applicator (108) contacts the surface.

### FIELD OF THE INVENTION

The present invention relates to a liquid applicator device to apply/paint liquid on a surface of a body part of a user.

### BACKGROUND OF THE INVENTION

Before performing a surgery by creating an aperture on the skin of a body part of a user, it is a pre-operative procedure to scrub the body part with soap and water, and thereafter, an antiseptic liquid is applied on the body part to disinfect/sterilize the body part and the area surrounding the body part. The main objective of performing the pre-operative procedure, is to ensure that microbial loads are not present on the skin of the body part before the surgery. There have been horrific incidents in the operation theatres of patients experiencing complications due to the presence of microbial loads while performing the surgery. Therefore, ensuring that the operation theater and the patient are completely sterilized before performing the surgery is a very essential step.

A common traditional practice while performing the pre-operative procedure is to dip cotton gauze/sponge or a cotton wool in the antiseptic liquid with the help of a holder and then apply the antiseptic liquid on the skin. This common traditional practice is disadvantageous in many ways due to the possibility of the excess antiseptic liquid dripping from the cotton gauze/sponge. Further, when a larger area of the body part is required to be applied with the antiseptic liquid, owing to the dripping of the antiseptic liquid, there are chances of the antiseptic liquid getting contaminated. Further, since the cotton gauze/sponge is just used along with the help of the holder, there is a possibility of uncontrolled amount of the antiseptic liquid being used that may be either insufficient or too much, which may get wasted.

Numerous improved devices and methods have been implemented to overcome the above indicated disadvantages of the common traditional practice such as providing with rupturable joints and glass vials any mechanical membranes. However, even these improved devices and methods either lack control of the delivery of the fluid to the sponge such that the dripping is avoided or are limited to the capacity of the sponge retaining the antiseptic liquid. This is due to, apart from manufacturing complexities to ensure control delivery of the liquid to the sponge, there are other challenges such as of reliability of rupturable joints or the risk with the breaking of glass vails or any mechanical membranes are very much possible.

In view of the above, there is a dire requirement for an efficient liquid applicator device which solves the above problems.

### SUMMARY OF THE INVENTION

One or more embodiments of the present disclosure provides a liquid applicator device to apply/paint liquid on a surface of a body part of a user.

In one aspect of the present invention, a method of a liquid applicator device to apply/paint liquid on a surface of a body part of a user is provided. The method includes a step of coupling a first end of a storage unit with and a second end of a connecting unit. The method further includes a step of coupling a first end of the connecting unit to a top surface of an applicator. The method further includes a step of defining an opening though a surface of the connecting unit. The method further includes a step of coupling a regulating unit to the connecting unit via the opening to regulate flow of a liquid from the storage unit to the applicator. The method further includes a step of coupling a seal breaker to a first surface of a spigot of the regulating unit. In an embodiment, the seal breaker is adapted to pierce through a membrane of the storage unit upon movement of the storage unit towards the seal breaker.

In another aspect of the present invention a device is disclosed. The device includes a storage unit having a first end and a second end at a proximal distance from the first end, adapted to store liquid therein. Further, the system includes a connecting unit having a first end and a second end at a proximal distance from the first end, coupled to a first end of the storage unit. The system further includes an applicator having a top surface coupled to the first end of the connecting unit and a bottom surface adapted to be in contact with a body part of a user, adapted to one of absorb and retain the liquid for a predefined period. The system further includes a regulating unit having a spigot and one or more through holes defined thereon, positioned within an opening of the connecting unit, the regulating unit adapted to regulate a flow of the liquid from within the storage unit towards the applicator. The system further includes a seal breaker, coupled to a first surface of the spigot of the regulating unit, adapted to pierce through a membrane of the storage unit to allow flow of the liquid from the storage unit towards the applicator via the one or more through holes. The connecting unit and the storage unit is one of a single component and separate components. The second end of the storage unit is adapted to be one of temporarily and permanently sealed. The storage unit, the connecting unit, and the applicator is coupled to each other via one of threading, molding, and thermal coupling. The one or more through holes are adapted to one of break air gap, prevent air bubbles, and allow flow of the liquid from the storage unit towards the applicator. The flow of the liquid is regulated based on an actuation of the regulation unit in one of a clockwise and an anti-clockwise direction. The applicator is further configured to desorb liquid from a body part in response to contact between the bottom surface of the applicator and the body part. The applicator is one of a fibrous material, multilayer gauze cloth and synthetic foam. In an embodiment, the applicator is selected based on an application for which the device is used for. The storage unit includes a membrane defined at the first end of the storage unit, wherein the membrane of the storage unit is adapted to come in contact with the seal breaker in response to a movement of the storage unit towards the seal breaker. The seal breaker is one of, a wedge shape head, a diamond shape head and a cylindrical shape head. The opening is defined adjacent to the first end of the storage unit.

Other features and aspects of this invention will be apparent from the following description and the accompanying drawings. The features and advantages described in this summary and in the following detailed description are not all-inclusive, and particularly, many additional features and advantages will be apparent to one of ordinary skill in the relevant art, in view of the drawings, specification, and claims hereof. Moreover, it should be noted that the language used in the specification has been principally selected for readability and instructional purposes and not have been selected to delineate or circumscribe the inventive subject matter, resort to the claims being necessary to determine such inventive subject matter.

### BRIEF DESCRIPTION OF THE DRAWINGS

The accompanying drawings, which are incorporated herein, and constitute a part of this disclosure, illustrate exemplary embodiments of the disclosed methods and systems in which like reference numerals refer to the same parts throughout the different drawings. Components in the drawings are not necessarily to scale, emphasis instead being placed upon clearly illustrating the principles of the present disclosure. Some drawings indicates the components using block diagrams and possibly not represent the internal circuitry of each component.

FIG. 1 illustrates a liquid applicator device, according to various embodiments of the present disclosure;

FIG. 2 illustrates a sectional view of a liquid applicator device of FIG. 1, according to various embodiments of the present disclosure;

FIG. 3 illustrates a liquid applicator device with a detached storage unit, according to various embodiments of the present system.

FIG. 4 is a flow diagram illustrating a method for providing a liquid applicator device, according to one or more embodiments of the present disclosure.

The foregoing shall be more apparent from the following detailed description of the invention.

### DETAILED DESCRIPTION OF THE INVENTION

Some embodiments of the present disclosure, illustrating all its features, will now be discussed in detail. It must also be noted that as used herein and in the appended claims, the singular forms “a”, “an” and “the” include plural references unless the context clearly dictates otherwise.

Various modifications to the embodiment will be readily apparent to those skilled in the art and the generic principles herein are applied to other embodiments. However, one of ordinary skill in the art will readily recognize that the present disclosure including the definitions listed here below are not intended to be limited to the embodiments illustrated but is to be accorded the widest scope consistent with the principles and features described herein.

A person of ordinary skill in the art will readily ascertain that the illustrated steps detailed in the figures and here below are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

As per various embodiments depicted, the present invention discloses the liquid applicator device to apply/paint liquid on a surface of a body part of a user. The liquid applicator device of the present invention is mainly used during the pre-operative procedure. The pre-operative procedure includes first washing the body part with soap and water. Thereafter, using the liquid stored within the liquid applicator device, here the liquid stored within the liquid applicator device is an antiseptic liquid and the antiseptic liquid is applied on the body part in a controlled manner.

In alternate embodiments, the liquid applicator device can be used to apply liquid on any given surface for applications other than the pre-operative procedure.

An object of the present invention is to ensure removal of microbial loads on the body part of the user.

Another object of the present invention is to apply paint on the body part of the user.

Yet another object of the present invention to ensure that delivery of liquid onto a foam of the liquid applicator device happens in a controlled manner.

Yet another object of the present invention is to ensure that excess amount of the liquid is not absorbed by the foam of the liquid applicator device so that the dripping of the liquid from the foam of the liquid applicator device is prevented.

Yet another object of the present invention is to ensure that the that the liquid applied by the liquid applicator device on a body part is not in excess and thereby not wasted.

Yet another object of the present invention is to ensure that the liquid applicator device can be customized to retain liquid of different capacities.

Yet another object of the present invention is to ensure that the air traps are prevented, thereby allowing consistent flow of liquid to a foam pad of liquid applicator.

Yet another object of the present invention is to ensure that the liquid applicator device is compact and simple to use with minimum components yet providing a controlled delivery of liquid to a surface, but not limited to the surface of the body part.

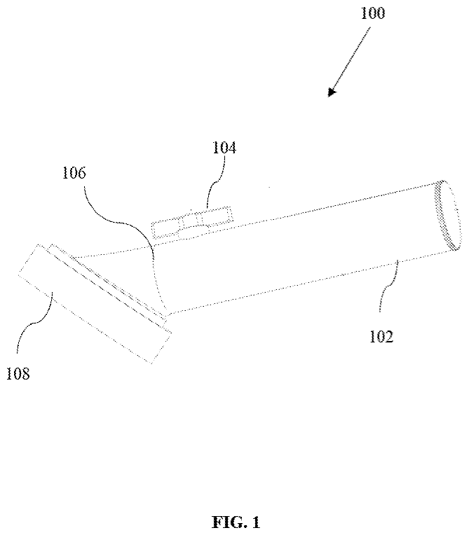

Referring to FIG. 1, FIG. 1 illustrates a liquid applicator device 100 according to one or more embodiments of the present invention. In the preferred embodiments, the liquid applicator device 100 includes a storage unit 102, a regulating unit 104, a connecting unit 106 and an applicator 108.

In an embodiment, the storage unit 102 is a cylindrical container adapted to store the liquid therein. In an alternate embodiment, the storage unit 102 is a barrel, a bottle or any other similar structure, but not limiting the scope of the present invention. Depending on the various applications of the liquid applicator device 100, the shapes, and the sizes of the storage unit 102 are different.

In an embodiment, the storage unit 102 is permanently attached with the connecting unit 106.

In an alternate embodiment, the storage unit 102 is detachable from the connecting unit 106.

In an embodiment, the capacity of the storage unit 102 varies depending on the requirements and the application for which the applicator 108 are used for. The capacity of the storage unit 102 ranges from about 3 to 100 millilitre (ml). However, the range of capacity should not be construed as limiting the scope of the present invention.

In an embodiment, the storage unit 102 comprises of a chamber (not shown).

In an embodiment, the chamber is detachable from the storage unit 102, thereby facilitating in removing the liquid from the storage unit 102. Alternatively, the chamber is permanently fixed within the storage unit 102.

In an embodiment, the liquid stored in the storage unit 102 includes one of, but not limited to, an antiseptic liquid, paint, water, and gel. For example, the antiseptic liquids include at least one of, but not limited to, alcohol, chlorhexidine Gluconate, Povidone Iodine, Hypochlorous acid, Hydrogen Peroxide, Benzalkonium Chloride or the combination thereof.

In an embodiment, the storage unit 102, the connecting unit 106 and the applicator 108 are coupled to each other via at least one of, but not limited to, threading, molding, and thermal coupling.

In an alternate embodiment, the connecting unit 106 and the storage unit 102 is one of a standalone component.

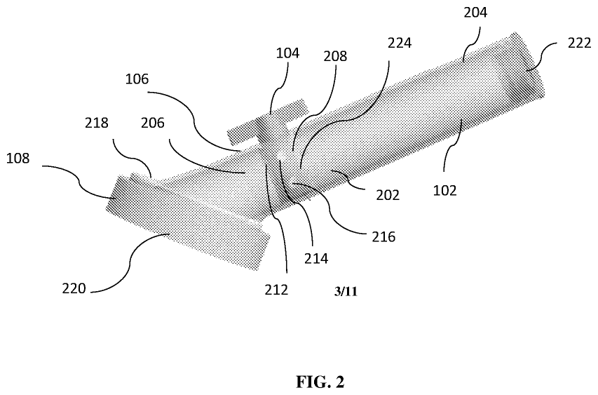

In an embodiment, an opening 210 is provided on a surface of the connecting unit 106 and adjacent to the storage unit 102 as shown in FIG. 2. The regulating unit 104 is positioned within the opening 210 of the connecting unit 106. The regulating unit 104 is adapted regulate a flow of the liquid within the storage unit 102 towards the applicator 108. In an embodiment, the regulating unit 104 is one of, but not limited to, a head, a valve, and a button. In order to regulate the flow of the liquid from the storage unit 102 towards the applicator 108, the regulating unit 104 is actuated in a predefined direction.

In an embodiment, the applicator 108 is adapted to absorb the liquid received from the storage unit 102 through the regulating unit 104 and retain the absorbed liquid for a predefined time period. The applicator 108 is further adapted to be in contact with the surface of the body part of a user. The applicator 108 is one of, but not limited to a fibrous material, multilayer gauze cloth and synthetic foam. In an alternate embodiment, the applicator 108 is selected based on an application for which the liquid applicator device 100 is used for.

Operational and construction features of the liquid applicator device 100 will be explained in detail with respect to the following figures.

Referring to FIG. 2, FIG. 2 illustrates a sectional view of a liquid applicator device 100 of FIG. 1, according to various embodiments of the present invention.

As shown in FIG. 2, the storage unit 102 includes a first end 202 and a second end 204. The second end 204 of the storage unit 102 is at a proximal distance from the first end 202 of the storage unit 102. The second end 204 of the storage unit 102 is adapted to be one of temporarily and permanently sealed via one of, but not limited to, a cap 222.

Further, the second end 204 of the storage unit 102 is temporarily sealed. The second end 204 of the storage unit 102 is closed and opened using a cap 222. Advantageously, the cap 222 ensures the liquid stored in the storage unit 102 is leakproof. Due to threading the cap 222 to the storage unit 102 advantageously ensures optimum pressure and air balance are maintained within the storage unit 102.

In an embodiment, the connecting unit 106 includes a first end 206 and a second end 208. The second end 208 of the connecting unit 106 is at a proximal distance from the first end 206 of the connecting unit 106. The second end 208 of the connecting unit 106 is coupled with the first end 202 of the storage unit 102 by one of, but not limited to threading, molding, and thermal coupling.

In an embodiment, the connecting unit 106 further includes an opening 210. The opening 210 is defined adjacent to the first end of the storage unit 202. However, a position of the opening 210 adjacent to the first end of the storage unit 202 should not be construed as limiting the scope of the present invention as the opening 210 may be located in any other section of the connection unit 106. The regulating unit 104 is positioned within an opening 210 on the surface of the connecting unit 106.

In an embodiment, the regulating unit 104 is adapted to regulate the flow of the liquid from the storage unit 102 towards the applicator 108 in a controlled manner. The flow of the liquid is regulated based on an actuation of the regulation unit 104 in one of a clockwise and an anti-clockwise direction. The regulating unit 104 includes a spigot 212, a first through hole 214 and a second through hole 216. In an alternate embodiment, the spigot 212 includes a first surface 224 on which the first through hole 214 and the second through hole 216 are present. The regulating unit 104 includes plurality of through holes that enables better liquid flow control and liquid flow management. The first through hole 214 is adapted to break air gap and prevent entrapment of air bubbles. The second through hole 216 is adapted to allow liquid to passthrough from the storage unit 102 to the applicator 108. In view of the this, having a spigot 212 should not be construed as limiting the scope of the present invention to just one spigot as the present invention is adapted to accommodate multiple spigots for one or more through holes to achieve the various objects of the present invention. Fundamentally, the two through holes 214, 216 are a big boon for the liquid applicator device since the two through holes 214, 216 regulate air pressure and allows liquid to flow from the storage unit 102 to the applicator 108. The two through holes 214, 216 size varies from about 3 mm to 7 mm.

In an embodiment, the two through holes 214, 216 are of the same diameter.

In an alternate embodiment, the two through holes 214, 216 may be of different diameters, thereby the through hole 216 with the bigger diameter compared to the other through hole 214 will facilitate the flow of liquid therethrough from the storage unit 102 to the applicator 108. Further, the through hole 214 with the smaller diameter will facilitate the flow of air therethrough in a direction opposite to the direction of flow of the liquid of the through hole 216 with the larger diameter.

In an embodiment, the position of the first through hole 214 and the second through hole 216 could be one of, but not limited to, first through hole 214 being in a raised position compared to the second through hole 216 in relation to the storage unit 102. However, these positions of the first through hole 214 and the second through hole 216 should not be construed as limiting the scope of the present invention, as the first through hole 214 and the second through hole 216 can be designed at other positions as well to ensure that air bubbles are prevented.

In an embodiment, with reference to the geometry of the first through hole 214 and the second through hole 216, the diameter of the first through hole 214 is greater compared to the second through hole 216. However, this geometry of the first through hole 214 and the second through hole 216 should not be construed as limiting the scope of the present invention, as the first through hole 214 and the second through hole 216 can be designed to have different geometry as well to ensure that air bubbles are prevented.

In an embodiment, the applicator 108 is a liquid permeable membrane which includes a top surface 218 and a bottom surface 220. The applicator 108 is permanently or detachably coupled to the connecting unit 106. The top surface 218 of the applicator 108 is coupled to the first end 206 of the connecting unit 106. The bottom surface 220 of the applicator 108 is adapted to be in contact with the surface of the body part of a user. The applicator 108 is further adapted to absorb and retain the liquid for a predefined time period. The applicator 108 is further configured to desorb the liquid on a body part in response to contact between the bottom surface 220 of the applicator 108 and the body part of the user.

In an alternate embodiment, the applicator 108 is detachably coupled to the connecting unit 106. Depending on the application for which the applicator 108 is used for, the applicator 108 is selected from the plurality of applicators 108 available in different shapes, sizes and colours.

In an alternate embodiment, the applicator 108 is selected based on one or more parameters such as, but not limited to, material, thickness, retention capacity of the liquid depending on the application for which the applicator 108 is used for.

In an embodiment, when the liquid applicator device 100 is in a non-operating state, the regulating unit 104 is in a closed position. In the closed position, the regulating unit 104 is perpendicular with reference to the storage unit 102. As the regulating unit 104 is in the closed position, the spigot 212, the first through hole 214 and the second through hole 216 are closed, thereby ensuring that the liquid is contained within the storage unit 102 and is not transferred to the applicator 108.

In an alternate embodiment, the liquid applicator device 100 is adapted to change its state from the non-operating state to an operating state, when the regulating unit 104 is actuated by a user by turning the regulating unit 104 in the clockwise and the anti-clockwise direction. Subsequent to turning the regulating unit 104 the clockwise or the anti-clockwise direction, the regulating unit 104 aligns at zero degrees with reference to the storage unit 102 and due to this actuation, the spigot 212, the first through hole 214 and the second through hole 216 are opened. During the course of operation, the liquid tends to rush through at least one of, the first through hole 214 and the second through hole 216, however, due to the position of the first through hole 214 and the second through hole 216, and the geometry of the first through hole 214 and the second through hole 216, the equilibrium between the first through hole 214 and the second through hole 216, the air flow control is attained and hence the air liquid is free to flow from the first through hole 214 and the liquid is free to flow from the second through hole 216 towards the liquid applicator 108.

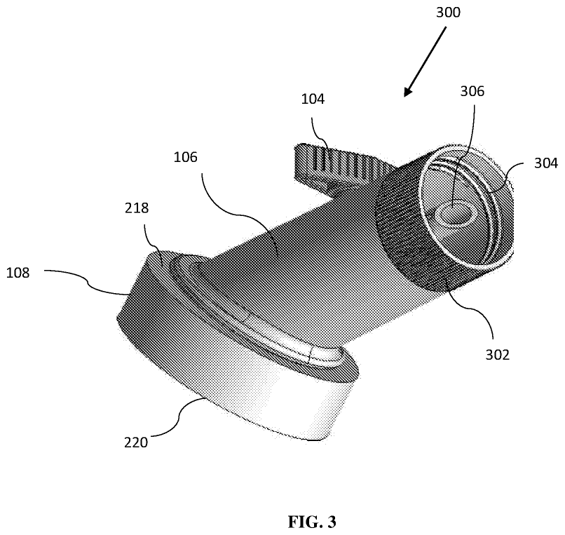

Referring to FIG. 3, FIG. 3 describes liquid applicator device 300 with detachable storage unit 102, according to various embodiments of the present system. It is to be noted that the embodiment with respect to FIG. 3 will be explained with a detachable storage unit 102 for the purpose of description and illustration and should nowhere be construed as limited to the scope of the present disclosure.

In an embodiment, based on the requirement of the quantity of a liquid in various applications, the storage unit 102 can be detached from the liquid applicator device 300 and replaced with the alternate storage unit 102 including at least one of, but not limited to, a barrel, a bottle, and a container. In an alternate embodiment, the liquid applicator device 300 may be used as a disposable product by using a new liquid applicator device 300 each time with the same bottle for different users.

In an embodiment, the liquid applicator device 300 without the storage unit 102 as shown in FIG. 3 may be directly coupled to the mouth of the bottle (an alternative to the storage unit 102) by screwing a cap 302 provided on the device 300 using one or more coupling mechanism 304, but not limited to threading. The threading 304 is provided on the inner surface of the second end 208 of the connecting unit 106. The threading 304 provided on the inner surface of the second end 208 of the connecting unit 106 is universally compatible with the threading provided on most of the bottles containing the liquid. In alternate embodiments, the connecting unit 106 is coupled to the bottle containing the liquid using other coupling means as well.

In an embodiment, the liquid applicator device 300 includes the regulating unit 104 coupled with the connecting unit 106.

In an embodiment, the liquid applicator device 300 further includes a seal breaker 306 which is coupled to the first surface 224 of the spigot 212 of the regulating unit 104. In an embodiment, the seal breaker 306 is at least one of, but not limited to, a wedge shape head, a diamond shape head and a cylindrical shape head. The seal breaker 306 is adapted to pierce through a membrane of the storage unit 102 to allow flow of the liquid from the storage unit 102 towards the applicator 108 via the first through hole 214 and the second through hole 216. In an alternate embodiment, the seal breaker 306 is coupled to the first surface 224 of the spigot 212 and between the first through hole 214 and the second through hole 216. However, the coupling position of the seal breaker 306 should not be construed as limiting the scope of the present invention. Subsequent to piercing the membrane of the storage unit 102, the regulating unit 104 is actuated that initiates the flow of liquid towards the applicator 108 in a controlled manner. Advantageously, usage of the seal breaker 306 provided on the first surface 224 of the spigot 212 of the regulating unit 104 ensures that the user operating the liquid applicator device 300 is not required to touch the mouth of the bottle, thereby avoid any kind of contamination of the liquid contained in the storage unit 102.

In an embodiment, the liquid applicator device 100 is capable of operating as a standalone device without the requirement of the seal breaker 306 when the storage unit 102 is permanently attached to the liquid applicator device 100. Further, in another embodiment, as shown in FIG. 3, the liquid applicator device 300 is capable of operating as a standalone device with the mechanism of the seal breaker 306 when the storage unit is detachably coupled to the liquid applicator device 300.

An exemplary working example of the liquid applicator device 100, 300 is illustrated below. It is to be noted that the working example is purely exemplary in nature and should nowhere be construed as limiting the scope of the present invention.

At the outset, the liquid applicator device 100 is required to be sterilized by any appropriate methods in clinical setup like Gamma Irradiation, ETO, gas fumigation or conventional steam sterilization.

When the liquid applicator device 100 is required to be used, the user is required to remove the outer package and then carefully open the liquid applicator device 100 in a sterile environment. Thereafter, the user actuates the regulating unit 104 by turning the regulating unit 104 in the pre-defined direction, preferably either in a clockwise or anti-clockwise direction. Subsequent to turning the regulating unit 104, the spigot 212, the first through hole 214 and the second through hole 216 are opened. As soon as the regulating unit 104 is actuated, the first through hole 214 is adapted to break the air gap and thereby air bubble entrapment in the storage unit 102 is avoided. Further, the cap 222 which closes the second end 204 of the storage unit 102 also ensures that optimum pressure and air balance are maintained within the storage unit 102, when the spigot 212 is opened.

In an embodiment, subsequent to actuating the regulating unit 104, the liquid starts flowing through the at least one of, the first through hole 214 and the second through hole 216 of the regulating unit 104 and then from the first end 206 of the connecting unit 106, the liquid reaches the top surface 218 of the applicator 108. In an alternate embodiment, the first through hole 214 is configured to control the air flow and the second through hole 216 is configured to control the liquid flow from the storage unit 102 to the applicator 108. Once the applicator 108 such as the synthetic foam is soaked with the liquid, the liquid applicator device 100 is ready to use and the liquid is applied on the surface of the body part of the user via the bottom surface 220 of the applicator 108 as the bottom surface 220 desorbs the liquid. Depending on the nature of the application, area of body part to be coated with the liquid, and also the number of layers of liquid to be coated, the liquid regulating unit 104 is adjusted to allow appropriate amount of liquid to flow through the regulating unit 104 to the applicator 108.

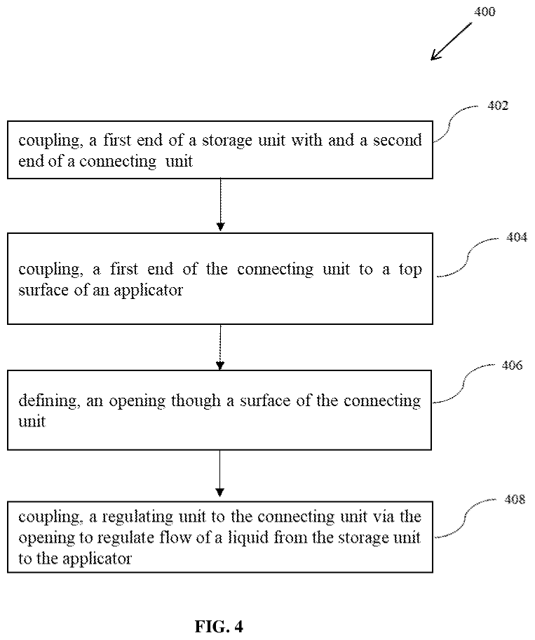

FIG. 4 shows a flow diagram of a method 400 for providing a liquid applicator device, according to various embodiments of the present disclosure. For the purpose of description, the method 400 is described with the embodiments as illustrated in FIG. 2 and should nowhere be construed as limiting the scope of the present disclosure.

At step 402, the method 400 includes the step of coupling a first end 202 of a storage unit 102 with and a second end 208 of a connecting unit 106. In an embodiment, when the liquid applicator device 100 is actuated, the liquid stored within the storage unit 102 flows from the first end 202 of the storage unit 102 towards the second end 208 of the connecting unit 106. In an embodiment, the storage unit 102 contains liquid to be applied on the surface of the body part of the user. In an embodiment, the first end 202 of the storage unit 102 is coupled with the second end 208 of the connecting unit 106 via at least one of, but not limited to, threading, molding, and thermal coupling.

At step 404, the method 400 includes the step of coupling, a first end 206 of the connecting unit 206 to a top surface 218 of an applicator 108. In an embodiment, when the liquid applicator device 100 is actuated, the first end 206 of the connecting unit 206 allows the flow of liquid towards the top surface 218 of an applicator 108 by passing through the spigot 212, the first through hole 214 and the second through hole 216. In an embodiment, the top surface 218 of the applicator 108 is coupled with the first end 206 of the connecting unit 106 via at least one of, but not limited to, threading, molding, and thermal coupling. In an embodiment, the top surface 218 of the applicator 108 is adapted to absorb the liquid received from the first end 206 of the connecting unit 106 and retain the absorbed liquid for a predefined time period.

At step 406, the method 400 includes the step of defining, an opening 210 though a surface of the connecting unit 106. In an embodiment, the opening 210 allows the positioning of the regulating unit 104 in the liquid applicator device 100. The position of the opening 210 is not limited to the top surface of the connecting unit 106. The opening 210 is also defined adjacent to the first end 202 of the storage unit 202.

At step 408, the method 400 includes the step of coupling, a regulating unit 104 to the connecting unit 106 via the opening 210 to regulate flow of a liquid from the storage unit 102 to the applicator 108. The regulating unit 104 is positioned within an opening 210 on the surface of the connecting unit 106. In an embodiment, the liquid applicator device 100 is actuated by actuating the regulating unit 104. Based on the actuated liquid applicator device 100, the regulating unit 104 is adapted to regulate the flow of the liquid from the first end 206 of the storage unit 102 towards the bottom surface 220 of the applicator 108 in a controlled manner. Initially, the flow of liquid starts from the first end 206 of the storage unit 102 to the second end 208 of the connecting unit 106. Then the liquid passes through the regulating unit 104 including the spigot 212, the first through hole 214 and the second through hole 216. Further, from the regulating unit 104 the liquid reaches to the first end 206 of the connecting unit 106. Further, from the first end 206 of the connecting unit 106 the liquid reaches to the top surface 218 of the applicator 108 which absorbs and retains the liquid for a predefined time period. Further, when the bottom surface 220 of the applicator 108 contacts with the surface of the body part of the user, the bottom surface 220 of the applicator 108 desorbs the liquid on the body part of the user.

The method 400 further includes the step of coupling, a seal breaker 306 to a first surface 224 of a spigot 212 of the regulating unit 104, wherein the seal breaker 306 is adapted to pierce through a membrane of the storage unit 102 upon movement of the storage unit 102 towards the seal breaker 306. In an embodiment, the seal breaker 306 is permanently or detachably coupled to the first surface 224 of the spigot 212 of the regulating unit 104 via at least one of, but not limited to, threading, molding, and thermal coupling. Further the storage unit 102 is detachably coupled with connecting unit 106 via a cap 302 with the one or more coupling mechanism, but not limited to threading. The storage unit 102 includes the membrane at its first end 202. While coupling the storage unit 102 with the connecting unit 106, the first end 202 of the storage unit 102 moves towards the seal breaker 306 and the seal breaker 306 is adapted to pierce through the membrane at the first end 202 of the storage unit 102 when the storage unit 102 is coupled with connecting unit 106.

A person of ordinary skill in the art will readily ascertain that the illustrated embodiments and steps in description and drawings (FIG. 1-4) are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

The present disclosure incorporates technical advancement of delivering of the liquid onto a foam pad/sponge in a controlled manner. The liquid applicator device ensures that dripping of liquid from the pad/sponge is prevented. The liquid applicator device prevents microbial load on the body part of the user. The liquid applicator device ensures that the liquid applied by the liquid applicator device on a body part is not in excess and thereby not wasted. The liquid applicator device ensures that the liquid applicator device is compact and simple to use with minimum components yet providing a controlled delivery of liquid to a surface. The liquid applicator device ensures that the liquid applicator device can be used as per the requirement throughout the body part irrespective of the area of the surface of the body part. The body part is at least one of a small surface area and a large surface area. The liquid applicator device ensures that the liquid applicator device can be customized to retain liquid of different capacities. The liquid applicator device ensures that the air traps are prevented, thereby allowing consistent flow of liquid to a liquid applicator such as a foam pad.

The present invention offers multiple advantages over the prior art and the above listed are a few examples to emphasize on some of the advantageous features. The listed advantages are to be read in a non-limiting manner.

### REFERENCE NUMERALS

Liquid Applicator device—100;
Storage unit—102;
Regulating unit—104;
Connecting unit—106;
Applicator—108;
First end of the storage unit—202;
Second end of the storage unit—204;
First end of the connecting unit—206;
Second end of the connecting unit—208;
Opening—210;
Spigot—212;
First through hole—214;
Second through hole—216;
Top surface of the applicator—218;
Bottom surface of the applicator—220;
Cap—222;
First surface of the spigot—224;
Cap—302;
One or more coupling mechanism—304;
Seal breaker—306;

We claim:

1. A device (100), comprising:
a storage unit (102), having a first end (202) and a second end (204) at a proximal distance from the first end (202), the storage unit adapted to store liquid therein;
a connecting unit (106), having a first end (206) and a second end (208) at a proximal distance from the first end (206), the second end (208) of the connecting unit is coupled to the first end (202) of the storage unit (102);
an applicator (108), having a top surface (218) coupled to the first end (206) of the connecting unit (106) and a bottom surface (220) adapted to be in contact with a body part of a user, the applicator (108) adapted to one of absorb and retain the liquid for a predefined period; and
a regulating unit (104), having a spigot (212) and one or more through holes (214, 216) defined thereon, the regulating unit (104) positioned within an opening (210) of the connecting unit (106), the regulating unit (104) adapted to regulate a flow of the liquid from within the storage unit (102) towards the applicator (108).

2. The device as claimed in claim 1, wherein the second end (204) of the storage unit (102) is adapted to be one of temporarily or permanently sealed.

3. The device as claimed in claim 1, wherein the one or more through holes (214, 216) are adapted to one of control airflow and allow flow of the liquid from the storage unit (102) towards the applicator (108).

4. The device as claimed in claim 1, wherein the flow of the liquid is regulated based on an actuation of the regulation unit (104) in one of a clockwise and an anti-clockwise direction.

5. The device as claimed in claim 1, wherein the applicator (108) is configured to desorb liquid from a body part in response to contact between the bottom surface (220) of the applicator (108) and the body part, wherein the applicator (108) is one of a fibrous material, multilayer gauze cloth and synthetic foam, the applicator (108) is selected based on an application for which the device is used for.

6. The device as claimed in claim 1, wherein the opening (210) is defined adjacent to the first end (202) of the storage unit (102).

7. The device as claimed in claim 1, wherein the device (100) further comprises:
a seal breaker (306), coupled to a first surface (224) of the spigot (212) of the regulating unit (104), adapted to pierce through a membrane of the storage unit (102) to allow flow of the liquid from the storage unit (102) towards the applicator (108) via the one or more through holes (214, 216), the seal breaker (306) is one of, a wedge shape head, a diamond shape head and a cylindrical shape head.

8. The device as claimed in claim 7, wherein the membrane of the storage unit (102) is defined at the first end (202) of the storage unit (102), wherein the membrane of the storage unit (102) is adapted to come in contact with the seal breaker (306) in response to a movement of the storage unit (102) towards the seal breaker (306).

9. A device (100), comprising:
a storage unit (102), having a first end (202) and a second end (204) at a proximal distance from the first end (202), adapted to store liquid therein;
a connecting unit (106), having a first end (206) and a second end (208) at a proximal distance from the first end, coupled to a first end (202) of the storage unit (102);
an applicator (108), having a top surface (218) coupled to the first end (206) of the connecting unit (106) and a bottom surface (220) adapted to be in contact with a body part of a user, adapted to one of absorb and retain the liquid for a predefined period;
a regulating unit (104), having a spigot (212) and one or more through holes (214, 216) defined thereon, positioned within an opening (210) of the connecting unit (106), the regulating unit (104) adapted to regulate a flow of the liquid from within the storage unit (102) towards the applicator

(108) ; and
a seal breaker (306), coupled to a first surface (224) of the spigot (212) of the regulating unit (104), adapted to pierce through a membrane of the storage unit (102) to allow flow of the liquid from the storage unit (102) towards the applicator (108) via the one or more through holes (214, 216).

10. A method for providing a device (100), comprising the steps of:
coupling, a first end (202) of a storage unit (102) with and a second end (208) of a connecting unit (106);
coupling, a first end (206) of the connecting unit (106) to a top surface (218) of an applicator (108);
defining, an opening (210) though a surface of the connecting unit (106); and
coupling, a regulating unit (104) to the connecting unit (106) via the opening (210) to regulate flow of a liquid from the storage unit (102) to the applicator (108).

11. The method as claimed in claim 10, wherein the method further comprising the step of:
coupling, a seal breaker (306) to a first surface (224) of a spigot (212) of the regulating unit (104), wherein the seal breaker (306) is adapted to pierce through a membrane of the storage unit (102) upon movement of the storage unit (102) towards the seal breaker (306).
